(19) United States
(12) Patent Application Publication
(10) Pub. No.: US 2019/0029561 A1
(43) Pub. Date: Jan. 31, 2019

(54) IMAGE REGISTRATION SYSTEM AND METHOD USING SUBJECT-SPECIFIC TRACKER

(71) Applicant: KOREA INSTITUTE OF SCIENCE AND TECHNOLOGY, Seoul (KR)

(72) Inventors: Hyung Min KIM, Seoul (KR); Hae Young JOE, Seoul (KR); Ki Joo PAHK, Seoul (KR); Inchan YOUN, Seoul (KR); Seung-Jong KIM, Seoul (KR)

(73) Assignee: KOREA INSTITUTE OF SCIENCE AND TECHNOLOGY, Seoul (KR)

(21) Appl. No.: 16/045,245

(22) Filed: Jul. 25, 2018

(30) Foreign Application Priority Data
Jul. 26, 2017 (KR) 10-2017-0094642

(51) Int. Cl. A61B 5/06; A61B 6/03; G06T 7/00

(52) U.S. Cl. CPC A61B 5/064; A61B 2090/3983; G06T 7/0014; A61B 6/032

(57) ABSTRACT

An image registration system using a subject-specific tracker includes: a photographing unit for obtaining a scanning image; a fabricated tracker based on anatomical data of the user including tracking markers for defining relative positions; a recognizing unit configured to recognize positions of the tracking markers and an image registration unit configured to register the scanning image based on the positions of the tracking markers. According to the system, a body part of the user and a scanning image may be automatically registered without a conventional image registration process such as an attachment of a fiducial marker and a confirmation of the fiducial marker position using CT/MRI imaging. Accordingly, it is possible to minimize an error caused by human, ensure precise registration, allow trackers to be attached or detached as necessary during surgery operation, and keep the accuracy of registration after the trackers are attached or detached.

### CROSS-REFERENCE TO RELATED APPLICATION

This application claims priority to Korean Patent Application No. 10-2017-0094642, filed on Jul. 26, 2017, and all the benefits accruing therefrom under 35 U.S.C. § 119, the contents of which in its entirety are herein incorporated by reference.

### BACKGROUND

### 1. Field

The present disclosure relates to an image registration system and method, and more particularly, to an image registration system and method for automatic registration of a scanning image of a user by using a customized tracker to match a body contour of the user.

### DESCRIPTION ABOUT NATIONAL RESEARCH AND DEVELOPMENT SUPPORT

This study was supported by the Brain Research Program through the National Research Foundation of Korea (NRF) Funded by the Ministry of Science, ICT & Future Planning (2015M3C7A1064833).

### 2. Description of the Related Art

Due to recent advances in medical technology, an image-guided surgery performed with an image of a patient obtained in advance by using a computed tomography (CT) device, a magnetic resonance imaging (MRI) device, a fluoroscope or an X-ray machine, is widely utilized. This surgery method is used in a case where an operator is difficult to see an affected part of a patient directly, such as brain nerve surgery or implant surgery, or should perform surgery while avoiding major nerves and organs in the body of the patient.

When a surgery is performed using a scanning image taken by a CT device or an MRI device, a registration process is performed to precisely match a coordinate of a scanning image of a patient and a coordinate of a real body of a patient by using a computer, and thus the operator may perform the surgery while watching the previously obtained scanning image on a monitor as if the operator views the body of the patient in real time.

The registration process is generally performed using a surgical navigation device. First, fiducial markers are attached to the body of a patient, and a three-dimensional scanning image is obtained using a CT device or an MRI device. Trackers are fixed around the fiducial markers, and relative positions between the fiducial markers and the trackers are measured from a device capable of recognizing the trackers to calculate a coordinate of the actual operation space. Also, the calculated coordinate is corresponded to the coordinate of the three-dimensional scanning image obtained in advance by using the CT device or the MRI device. Further, a coordinate of a surgical tool is corresponded to the coordinate of the scanning image, and the operator may perform the surgical operation while indirectly viewing the body of the user and the surgical tool, displayed on the monitor.

In a conventional method for matching a surgical space and an image space, if the positional relationship between the tracker and the patient changes during surgery, the registration process should be performed again during the surgical operation, which is troublesome and deteriorates the accuracy of the registration itself.

In order to solve this problem, a method of scanning a range to be operated by using a laser pointer scanner for registration of an image was developed. However, a coordinate may be input incorrectly, and a relatively expensive device should be used, which deteriorates the economic feasibility.

As another solution, U.S. Pat. No. 8,457,719 B2 proposes a registration method using a flexible substrate attachable to the face of a patient and LED tracking markers attached to the substrate, and a face coordinate of the patient and a coordinate of the surgical image are registered by calculating a position between the LED tracking markers. This method is cheaper than the method using the laser pointer scanner. However, since the flexible substrate is not user-customized, if the substrate moves or the position of the LED tracking marker is changed, the substrate should be registered again, and the accuracy may be changed during the new registration process.

### SUMMARY

The present disclosure is directed to providing an image registration system and method for registering a scanning image of a user by using customized trackers to match a body contour based on anatomical data of the user.

In detail, the present disclosure is directed to providing a technique for automatically registering a body part of the user and a scanning image without experiencing a conventional image registration process such as an attachment of a fiducial marker and a confirmation of the fiducial marker position using CT/MRI imaging. Accordingly, it is possible to minimize an error caused by human, ensure precise registration, allow trackers to be attached or detached as necessary during surgery operation, and keep the accuracy of registration after the trackers are attached or detached.

In one aspect, there is provided an image registration system using a subject-specific tracker, comprising: a photographing unit configured to obtain a scanning image of a user; a tracker fabricated based on anatomical data of the user and including a plurality of tracking markers for defining relative positions of body parts; a recognizing unit configured to recognize positions of the plurality of tracking markers; and an image registration unit configured to automatically register the scanning image of the user, based on the positions of the plurality of tracking markers.

In an embodiment, the photographing unit may be a computer tomography (CT) device or a magnetic resonance imaging (MRI) device.

In an embodiment, the tracker may include a customized mask to match a body contour of the user.

In an embodiment, the tracker may be customized by means of a 3D printing technique.

In an embodiment, the tracker may further include an implant guide framework or an ultrasonic focusing guide framework.

In an embodiment, the plurality of tracking markers may be made of a material which reflects a light of a specific wavelength, and the recognizing unit may include an optical camera capable of recognizing the light of a specific wavelength.

In an embodiment, the plurality of tracking markers may be LED elements.

In an embodiment, the plurality of tracking markers may be printed in a predetermined pattern design.

In another aspect of the present disclosure, there is also an image registration system using a subject-specific tracker, comprising: a photographing unit configured to obtain a scanning image of a user; an electromagnetic field generating unit configured to generate an electromagnetic field of a specific form; a plurality of electromagnetic field sensors fixed to a body of the user to recognize positions thereof by measuring the electromagnetic field generated from the electromagnetic field generating unit; a tracker fabricated based on anatomical data of the user and including the plurality of electromagnetic field sensors; and an image registration unit configured to register the scanning image of the user, based on the positions of the plurality of electromagnetic field sensors.

In another aspect of the present disclosure, there is also provided an image registration method using a subject-specific tracker, comprising: obtaining a scanning image of a user by using a photographing unit; recognizing positions of a plurality of tracking markers for defining relative positions of body parts by using a recognizing unit; and registering the scanning image of the user, based on the positions of the plurality of tracking markers, wherein the plurality of tracking markers are included in a tracker fabricated based on anatomical data of the user.

In an embodiment, the photographing unit may be a computer tomography (CT) device or a magnetic resonance imaging (MRI) device.

In an embodiment, the tracker may be a customized mask to match a body contour of the user.

In an embodiment, the tracker may be customized by means of a 3D printing technique.

In an embodiment, the tracker may further include an implant guide framework or an ultrasonic focusing guide framework.

In an embodiment, the plurality of tracking markers may be made of a material which reflects a light of a specific wavelength, and the recognizing unit may include an optical camera capable of recognizing the light of a specific wavelength.

In an embodiment, the plurality of tracking markers may be LED elements. In an embodiment, the plurality of tracking markers may be printed in a predetermined pattern design.

In another aspect of the present disclosure, there is also an image registration method using a subject-specific tracker, comprising: obtaining a scanning image of a user by using a photographing unit, generating an electromagnetic field of a specific form by using an electromagnetic field generator, recognizing positions of a plurality of electromagnetic field sensors by measuring the electromagnetic field generated from the electromagnetic field generator using the electromagnetic field sensors fixed to a body of the user and registering the scanning image of the user, based on the positions of the electromagnetic field sensors, wherein the electromagnetic field sensors are included in a tracker fabricated based on anatomical data of the user.

The image registration system using a subject-specific tracker according to an embodiment of the present disclosure uses a customized tracker to match a body contour of the body in order to register a coordinate of the body of the user and a coordinate of the scanning image. The tracker is fabricated to include a number of tracking markers for defining relative positions of body parts.

According to the system, there is a technique provided for automatically registering a body part of the user and a scanning image without experiencing a conventional image registration process such as an attachment of a fiducial marker and a confirmation of the fiducial marker position using CT/MRI imaging. Accordingly, it is possible to minimize an error caused by human, ensure precise registration, allow trackers to be attached or detached as necessary during surgery operation, and keep the accuracy of registration after the trackers are attached or detached.

Since the tracker can be customized using a 3D printing technique, its cost is low, and a framework or an ultrasonic focusing guide framework may be further included as necessary, thereby ensuring high extensibility as a medical device.

### BRIEF DESCRIPTION OF THE DRAWINGS

FIG. 1 is a diagram showing the image registration by corresponding a coordinate of a body of a user and a coordinate of an image.

FIG. 2 is a diagram showing the corresponding relationship of an automatic image registration system according to an embodiment.

FIG. 3 is a flowchart for illustrating each step of an automatic image registration method according to an embodiment.

### DETAILED DESCRIPTION

The present disclosure will be explained in detail with reference to the accompanying drawings which illustrate, by way of example, specific embodiments in which the present disclosure may be implemented. The embodiments are described in sufficient detail to enable those skilled in the art to implement the present disclosure. It should be understood that various embodiments of the present disclosure are different but need not be mutually exclusive. For example, specific features, structures and characteristics described herein may be implemented in other embodiments without departing from the scope of the present disclosure in connection with one embodiment. It should also be understood that the positions or arrangements of individual components in each embodiment can be varied without departing from the scope of the present disclosure. Therefore, the following detailed description is not intended to limit the present disclosure, and the scope of the present disclosure should be limited only by the appended claims, along with the full scope of equivalents to which the claims are entitled. In the drawings, like reference numerals denote the same or similar functions throughout the several aspects.

Hereinafter, preferred embodiments of a subject-specific automatic image registration system will be described in detail.

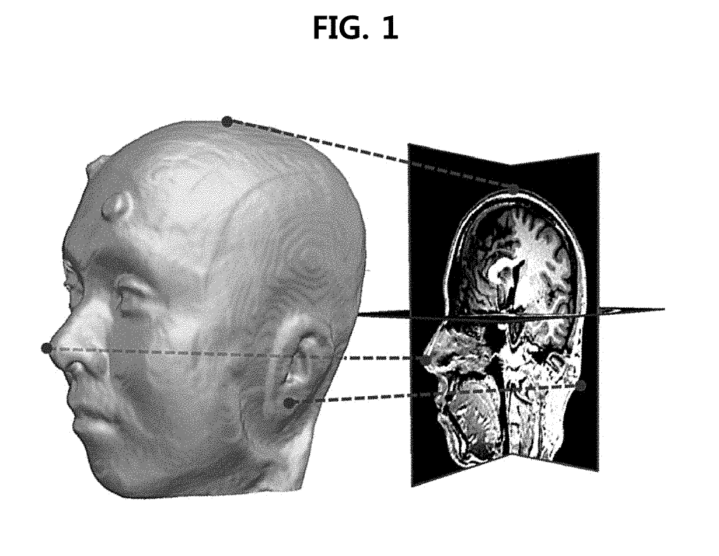

FIG. 1 is a diagram showing the image registration by corresponding a coordinate of a body of a user and a coordinate of an image. When an operator performs a surgical operation while watching a scanning image obtained by a photographing device, a registration process is performed to accurately match a coordinate of the scanning image of a patient that was photographed in advance as in a right side on the figure with a coordinate of an actual body of the patient as in a left side on the figure by using a computer. By doing so, the operator may perform the surgical operation while watching the scanning image photographed in advance as if the operator watches the body of the patient in real time.

The photographing device may include a computer tomography (CT) device or a magnetic resonance imaging (MRI) device, commonly used to obtain a three-dimensional scanning image, without being limited thereto. Here, the three-dimensional scanning image includes images obtained by manipulating or reconstructing an image directly photographed by a CT device or an MRI device, and also includes a multi-plane reconstructed image and a three-dimensional reconstructed image which are utilized for actual surgery.

According to a conventional image registration method, during brain surgery, one or more fiducial markers are attached to the face or skull of a patient. A three-dimensional scanning image is taken using a CT device or MRI device, then a tracker is fixed to a position spaced apart from the fiducial marker, and relative positions between the fiducial marker and the tracker are recognized by a device capable of recognizing the position of the tracker. After that, the image registration device (for example, a medical computer) is used to register a surgical space and an image space by mapping the position of the fiducial marker observed in the three-dimensional scanning image of a patient which was previously photographed using a photographing device (for example, a CT device or an MRI device) with the position of a fiducial marker tracked by the recognizing device by means of coordinate, and to display the location of the tracker on the monitor in real time.

According to the conventional method, since the fiducial markers should keep adhered, and if the positional relationship between the tracker and the patient changes during the surgery, the registration process should be performed again during the operation, which is troublesome and deteriorates the accuracy of the registration itself.

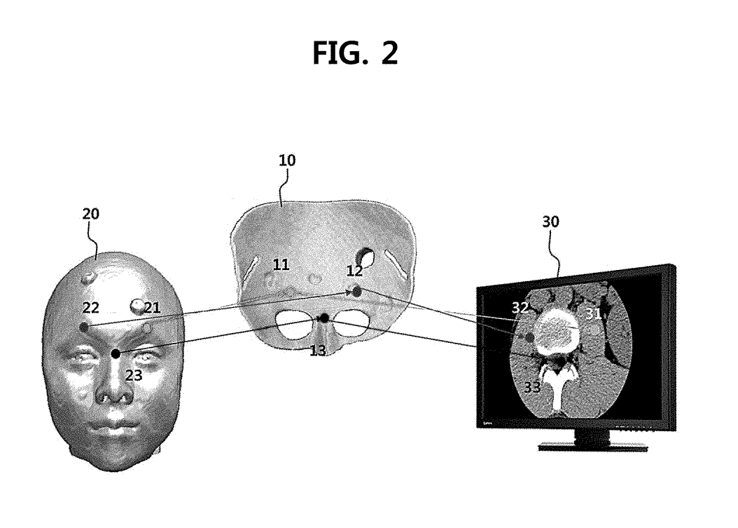

FIG. 2 shows an automatic image registration system for solving the above problem. FIG. 2 shows the corresponding relationship of an automatic image registration system according to an embodiment.

The system includes a photographing device for obtaining a scanning image of a user body (for example the face or the head) 20, a tracker 10 having a plurality of tracking markers for defining relative positions of body parts, a recognizing unit for recognizing the positions of the tracking markers, and an image registration unit 30 for registering the scanning image of the user based on the positions of the tracking markers.

Though not shown in the figures, the photographing unit includes a computer tomography (CT) device or a magnetic resonance imaging (MRI) device, which is generally used for obtaining a three-dimensional scanning image as described above. The photographed scanning image may be displayed on a monitor in the image registration device 30 after a registration process, explained later.

In an embodiment, the tracker 10 may be customized to match a body contour of the user (for example, in a mask form that is customized to match the face contour of the user) which may be fabricated using the 3D printing technique. The tracker 10 may be a guide framework such as a customized substrate or a patch to fit each part of the body using anatomical data of the user as well as a mask type that can be worn on the face, but is not limited to a form for a specific portion.

The 3D printer includes all devices that make a solid object based on a 3D drawing by adding motions in a front and back direction (x axis), in a left and right direction (y axis) and in an upper and lower direction (z axis), and may employ either a stack-type 3D printer that stacks up one layer by one layer or a cut-type 3D printer that cuts a single lump. However, in addition to the general 3D printing technique, any method capable of forming a tracker according to the body contour of the user may be used, and the present disclosure is not limited to a specific fabricating technique. In an embodiment, the tracker may be made of poly lactic acid (PLA) which is harmless to the human body, without being limited to any specific material.

As shown in FIG. 2, the tracker 10 may include a plurality of tracking markers 11 to 13. The tracker is fixed to a specific portion of the body to indicate the position and thus is used to define relative positions of the tracking markers in the tracker to body parts. Generally, the recognizing device serving as a reference records a relative position of the tracking marker fixed to the tracker and transmits coordinate information to a computer that performs registration.

In an embodiment of the present disclosure, the tracker may be fabricated using a 3D scanning photographing image of a patient. In this case, since the tracking marker is fixed at a desired position during a tracker fabricating process and the tracker coincides with the body contour of the user, the position of the tracking marker is not changed. Thus, registration is performed by simply putting the tracker including the tracking marker on the user.

The optical tracking marker may be largely classified into an active type and a passive type. The active-type tracking marker is configured such that each tracking marker displays its own position information, like an LED element, and the passive-type tracking marker is configured such that position information of each tracking marker is obtained by photographing the tracking marker by using an optical camera, like an element reflecting a light of a specific wavelength.

In an embodiment, a passive-type tracking marker made of a material reflecting a light of a specific wavelength or colored with the material to measure the position of the tracking marker using an optical camera capable of recognizing the light of the corresponding wavelength may be used, but the tracking method is not limited thereto. For example, an active-type tracking marker composed of LED elements may be inserted into the tracker.

As another example, in the case of a tracking system using an electromagnetic field, a magnetic field sensor may be used as the tracking marker. In this case, the magnetic field sensor may simultaneously function as a tracking marker and a recognizing unit for recognizing the magnetic field generated from the electromagnetic field generating device, and may track the position of the sensor from the electromagnetic field generating device. As another example, the plurality of tracking markers may be printed in a predetermined pattern design and tracked using the recognizing unit such as an optical camera.

Referring to FIG. 2, the plurality of tracking markers 11 to 13 included in the tracker 10 define relative positions of the specific portions 21 to 23 of the body 20 of the user. In an embodiment, an optical camera may be used to recognize each tracking marker made of a material that reflects a light of a specific wavelength. In this case, the optical camera measures the positions of the tracking markers 11 to 13 using a sensor capable of recognizing the reflected light of a specific wavelength.

The recognized position information of the tracking markers is transmitted to the image registration device 30, and the image registration device 30 matches coordinates 21 to 23 of the body parts defined by the tracking markers 11 to 13 and coordinates 31 to 33 of the three-dimensional scanning image photographed in advance to perform registration. For example, the tracking marker 11 corresponds to the body part 21 at the image coordinate 31, the tracking marker 12 corresponds to the body part 22 at the image coordinate 32, and the tracking marker 13 corresponds to the body part 23 at the image coordinate 33.

The image registration method of matching different space coordinates using a matrix is generally known and is not an essential technical idea of the present disclosure, and thus it is not described in detail here.

The shape of the tracker 10 and the position and number of the tracking markers 11 to 13 included in the tracker shown in FIG. 2 are just examples for better understanding, and the present disclosure is not limited to a specific shape, position or number. According to an embodiment, the shape of the tracker and the position and number of the tracking markers may be changed, and if the number of tracking markers increases, the range of space for recognizing the position of the tracker is widened. If the range of space for recognizing the position of the tracker is widened, the accuracy of image registration by position recognition may be improved. In addition, compared to a conventional registration technique using a flexible substrate and LED tracking markers, the tracker may be easily recognized with low cost since a desired number of tracking markers may be disposed at desired positions in the 3D printing process.

In an embodiment, the tracker may further include an implant guide framework or an ultrasonic focusing guide framework, which may be customized to the body contour of the user or a site to be treated by means of the 3D printing technique. In addition, the subject-specific tracker may have very high extensibility as a medical device, depending on the operation to be performed. For example, a subject-specific acoustic lens for low-intensity focusing ultrasonic treatment may be attached to the tracker to achieve high focusing efficiency, and simultaneously ultrasonic wave may be accurately collected to a desired focus by using the tracker.

If the image registration system described above is used, by the customized tracker based on the three-dimensional scanning image of the user, it is possible to automatically register the body part of the user and the scanning image without performing a conventional image registration process such as an attachment of a fiducial marker and a confirmation of the fiducial marker position using CT/MRI imaging. Accordingly, highly accurate registration is possible, and the patient may attach and detach the tracker as needed during surgical operation. Also, the accuracy of registration may be kept after attachment or detachment.

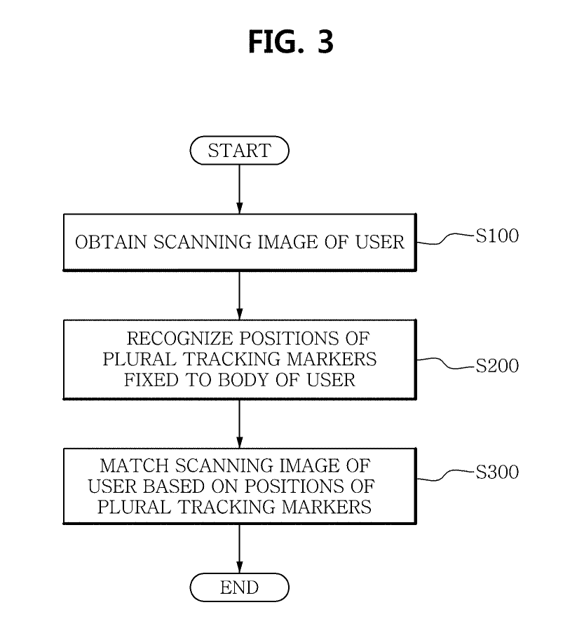

Hereinafter, an image registration method using a subject-specific tracker will be described with reference to FIG. 3.

First a scanning image of a user is obtained using a photographing unit (S100). As described above, the photographing device includes a computer tomography (CT) device or a magnetic resonance imaging (MRI) device, which is commonly used to obtain a three-dimensional scanning image, without being limited thereto.

Next, positions of a plurality of tracking markers for defining relative positions of the body parts of the user are recognized using a recognizing unit (S200). The plurality of tracking markers are included in a tracker, which are fabricated according to anatomical data of the user. In an embodiment, the tracker may have a mask form which may be printed using the 3D printing technique. The tracker may be made of poly lactic acid (PLA) which is harmless to the human body, but it is not limited to a specific material.

The tracker is fixed to a specific part of the body which is used to define relative positions of the tracking markers to body parts. As described above, the tracker is fixed at a desired position during a tracker fabricating process, and the tracker coincides with the body contour of the user so that the position of the tracking marker is not changed. Thus, registration is performed by simply putting the tracker including the tracking marker on the user.

In an embodiment, the tracker may further include an implant guide framework or an ultrasonic focusing guide framework. These frameworks may be customized to the body contour of the user or a site to be treated by means of the 3D printing technique. In addition, the subject-specific tracker may have very high extensibility as a medical device, depending on the operation to be performed. For example, a subject-specific acoustic lens for low-intensity focusing ultrasonic treatment may be attached to the tracker to achieve high focusing efficiency, and simultaneously ultrasonic wave may be accurately collected to a desired focus by using the tracker.

The plurality of tracking markers may be passive-type tracking markers made of a material reflecting a light of a specific wavelength, and in this case, may include an optical camera capable of recognizing the light of a specific wavelength.

As described above, the position and number of the tracking markers may be changed, and if the number of tracking markers increases, the range of space for recognizing the position of the tracker is widened. The kind and number of the tracking markers are not limited to a specific embodiment, and an active-type tracking marker composed of LED elements may be inserted into the tracker. In this case, the light emitted by the LED element may be recognized by the camera to measure the position of the tracking marker. For example, the tracking marker may be an LED tracking marker or printed in a specific pattern design. In this case, the recognizing unit may be a camera for recognizing the light or printed pattern of the tracking marker.

In addition, in the case of a tracking system using an electromagnetic field, a magnetic field sensor may be used as the tracking marker. In this case, the magnetic field sensor may simultaneously function as a tracking marker and a recognizing unit for recognizing the magnetic field generated from the electromagnetic field generating device, and may track the position of the sensor from the electromagnetic field generating device.

Next, the scanning image of the user may be registered based on the positions of the plurality of tracking markers (S300). The position information of the tracking marker recognized by the optical camera is transmitted to the image registration device, and registration is performed by matching the coordinate of the body part defined by the tracking marker with the coordinate of the three-dimensional scanning image photographed in advance.

As described above, the image registration method of matching different space coordinates using a matrix is generally known and is not an essential technical idea of the present disclosure, and thus it is not described in detail here.

If the image registration method is used with the customized tracker based on the three-dimensional scanning image of the user, it is possible to automatically register the body part of the user and the scanning image by putting the marker on the user without performing a conventional image registration process such as an attachment of a fiducial marker and a confirmation of the fiducial marker position using CT/MRI imaging. Accordingly, highly accurate registration is possible by minimizing an error caused by human, and the tracker may be attached or detached as needed during surgical operation. Also, the accuracy of registration may be kept after attachment or detachment.

Although the present disclosure has been described with reference to the embodiments shown in the drawings, it should be understood that various changes and modifications may be made thereto without departing from the scope of the present disclosure by those skilled in the art. However, such modifications should be considered to fall within the technical protection scope of the present disclosure. Accordingly, the true protection scope of the present disclosure should be defined by the technical idea of the appended claims.

What is claimed is:

1. An image registration system using a subject-specific tracker, comprising:
a photographing unit configured to obtain a scanning image of a user;
a tracker fabricated based on anatomical data of the user and including a plurality of tracking markers for defining relative positions of body parts;
a recognizing unit configured to recognize positions of the plurality of tracking markers and
an image registration unit configured to register the scanning image of the user, based on the positions of the plurality of tracking markers.

2. The image registration system using a subject-specific tracker according to claim 1,
wherein the photographing unit is a computer tomography (CT) device or a magnetic resonance imaging (MRI) device.

3. The image registration system using a subject-specific tracker according to claim 1,
wherein the tracker is customized to match a body contour of the user.

4. The image registration system using a subject-specific tracker according to claim 3,
wherein the tracker is customized by means of a 3D printing method.

5. The image registration system using a subject-specific tracker according to claim 4,
wherein the tracker further includes an implant guide framework or an ultrasonic focusing guide framework.

6. The image registration system using a subject-specific tracker according to claim 1,
wherein the plurality of tracking markers are made of a material which reflects a light of a specific wavelength, and
wherein the recognizing unit includes an optical camera capable of recognizing the light of a specific wavelength.

7. The image registration system using a subject-specific tracker according to claim 1,
wherein the plurality of tracking markers are LED elements.

8. The image registration system using a subject-specific tracker according to claim 1,
wherein the plurality of tracking markers are printed in a predetermined pattern design.

9. An image registration system using a subject-specific tracker, comprising:
a photographing unit configured to obtain a scanning image of a user;
an electromagnetic field generating unit configured to generate an electromagnetic field of a specific form;
a plurality of electromagnetic field sensors fixed to a body of the user to recognize positions thereof by measuring the electromagnetic field generated from the electromagnetic field generating unit;
a tracker fabricated based on anatomical data of the user and including the plurality of electromagnetic field sensors; and
an image registration unit configured to register the scanning image of the user, based on the positions of the plurality of electromagnetic field sensors.

10. An image registration method using a subject-specific tracker, comprising:
obtaining a scanning image of a user by using a photographing unit;
recognizing positions of a plurality of tracking markers for defining relative positions of body parts by using a recognizing unit and
registering the scanning image of the user, based on the positions of the plurality of tracking markers,
wherein the plurality of tracking markers are included in a tracker fabricated based on anatomical data of the user.

11. The image registration method using a subject-specific tracker according to claim 10,
wherein the photographing unit is a computer tomography (CT) device or a magnetic resonance imaging (MRI) device.

12. The image registration method using a subject-specific tracker according to claim 10,
wherein the tracker is customized to match a body contour of the user.

13. The image registration method using a subject-specific tracker according to claim 12,
wherein the tracker is customized by means of a 3D printing technique.

14. The image registration method using a subject-specific tracker according to claim 12,
wherein the tracker further includes an implant guide framework or an ultrasonic focusing guide framework.

15. The image registration method using a subject-specific tracker according to claim 10,
wherein the plurality of tracking markers are made of a material which reflects a light of a specific wavelength, and
wherein the recognizing unit is capable of recognizing the light of a specific wavelength.

16. The image registration method using a subject-specific tracker according to claim 10,
wherein the plurality of tracking markers are LED elements.

17. The image registration method using a subject-specific tracker according to claim 10,
wherein the plurality of tracking markers are printed in a predetermined pattern design.

18. An image registration method using a subject-specific tracker, comprising:
obtaining a scanning image of a user by using a photographing unit;
generating an electromagnetic field of a specific form by using an electromagnetic field generator;
recognizing positions of a plurality of electromagnetic field sensors by measuring the electromagnetic field generated from the electromagnetic field generator by using the electromagnetic field sensors fixed to a body of the user and
registering the scanning image of the user, based on the positions of the electromagnetic field sensors,
wherein the of electromagnetic field sensors are included in a tracker fabricated based on anatomical data of the user.
